(19) United States
(12) Patent Application Publication
(10) Pub. No.: US 2009/0081089 A1
(43) Pub. Date: Mar. 26, 2009

(54) Fluid reservoir and receiver assembly

(76) Inventors: Jeffrey G. Dayman, Waterloo (CA); Andy Kwan-Leung Sun, Toronto (CA)

Correspondence Address: CLARK & BRODY, 1090 VERMONT AVENUE, NW, SUITE 250, WASHINGTON, DC, 20005, US

(21) Appl. No.: 11/902,815

(22) Filed: Sep. 26, 2007

(51) Int. Cl. A61L 2/07

(52) U.S. Cl. 422/292

(57) ABSTRACT

There is provided a fluid supply assembly for providing a fluid or water supply to sterilization equipment. The assembly comprises a removable fluid reservoir and a receiver interface assembly for providing fluid from the reservoir to the sterilization equipment. The assembly may include a filter for treating the fluid supply.

### FIELD OF THE INVENTION

The present invention relates to a fluid reservoir and receiver assembly for use in an apparatus for sterilizing medical or dental instruments and the like.

### BACKGROUND OF THE INVENTION

Sterilization equipment, such as cassette autoclaves, typically require a supply of distilled water for creating steam during the sterilization process. A built-in water reservoir is used which is typically refilled with distilled water as needed. Such built-in water reservoirs may be difficult to access for cleaning. Improper cleaning of the reservoir leads to undesirable bio-film buildup in the reservoir. The water reservoir also may be difficult to access in order to refill, thus causing spillage during refilling or necessitating the use of a special container to refill the reservoir.

Further, since sterilization equipment typically requires the use of distilled or deionized water to prevent damage to certain components of the sterilization equipment, a supply of suitable water must be maintained or a separate device must be used to obtain distilled or deionized water.

In U.S. Pat. No. 6,793,900, Morck et al. disclosed the use of a separate water reservoir for use with sterilization equipment, so that the separate water reservoir may be easily cleaned or so that a disposable water reservoir may be used. Morck et al. also disclose a removable water reservoir but do not disclose the integration of a water receiver assembly and removable water reservoir in the sterilization device itself.

### SUMMARY OF THE INVENTION

Embodiments of the present invention provide a water receiver assembly and removable water reservoir for providing a water supply for sterilization equipment. By providing a removable water reservoir, the reservoir can be refilled at a tap or at a distilled water supply without the need for special refilling equipment. Leakage or spilling of water during the refilling process also is prevented. A removable water reservoir also enables convenient cleaning of the reservoir. In accordance with an embodiment of the present invention, the water receiver assembly and removable water reservoir may accept tap water, thus eliminating the need for a separate supply of distilled or deionized water for the sterilization equipment.

In accordance with an embodiment of the present invention, there is provided a fluid supply assembly for use in a sterilization apparatus. The assembly comprises a removable fluid reservoir having a fill means and an outlet port and an interface assembly having at least one inlet for receiving fluid from the reservoir and at least one outlet for directing fluid to the sterilization apparatus.

In accordance with another embodiment of the present invention there is provided a fluid supply assembly for use in a sterilization apparatus. The assembly comprises a removable fluid reservoir having a fill means and an outlet port, and a fluid cartridge having an inlet and an outlet. There is also provided an interface assembly having a first inlet for receiving fluid from the removable fluid reservoir, a first outlet for directing fluid to the fluid cartridge, a second inlet for receiving fluid from the fluid cartridge and a second outlet for directing fluid to the sterilization apparatus.

### BRIEF DESCRIPTION OF THE DRAWINGS

These and other advantages of the invention will become apparent upon reading the following detailed description and upon referring to the drawings in which:

FIG. 1 is a perspective view of a sterilization apparatus including a reservoir.

FIGS. 2A and 2B are perspective views of a removable reservoir in accordance with an embodiment of the invention.

FIG. 3 is a perspective view of a sterilization apparatus with the reservoir removed.

FIG. 4 is a perspective view of a sterilization apparatus with the reservoir and fluid cartridge removed.

FIG. 5 is a perspective view of a sterilization apparatus with the cover assembly and reservoir removed, showing a receiver assembly in accordance with an embodiment of the invention.

FIG. 6 is a perspective view of the front of a receiver assembly, separated from the sterilization apparatus.

FIG. 7 is a perspective view of the rear of a receiver assembly separated from the sterilization apparatus.

FIG. 8 is a cutaway view of a reservoir placed in a sterilization apparatus and mated with a receiver assembly.

FIG. 8A is a detailed cutaway view of the reservoir outlet and receiver assembly inlet of FIG. 8.

FIG. 9 is a cutaway view of a reservoir placed in a sterilization apparatus and mated with a receiver assembly.

FIG. 9A is a detailed cutaway view of the reservoir outlet and receiver assembly detection means of FIG. 9.

FIG. 10A is a side sectional view of a reservoir and fluid cartridge placed in a sterilization apparatus and mated with a receiver assembly.

FIG. 10B is a detailed side sectional view of the reservoir outlet and receiver assembly inlet of FIG. 10A.

FIG. 10C is a side sectional view of a reservoir placed in a sterilization apparatus and mated with a receiver assembly, with the fluid cartridge removed.

FIG. 10D is a detailed side sectional view of the reservoir outlet and receiver assembly inlet of FIG. 10C.

While the invention will be described in conjunction with the illustrated embodiments, it will be understood that it is not intended to limit the invention to such embodiments. On the contrary, it is intended to cover all alternatives, modifications and equivalents as may be included within the spirit and scope of the invention as defined by the appended claims.

### Detailed Description of the Preferred Embodiments

In the following description, similar features in the drawings have been given identical reference numerals where appropriate.

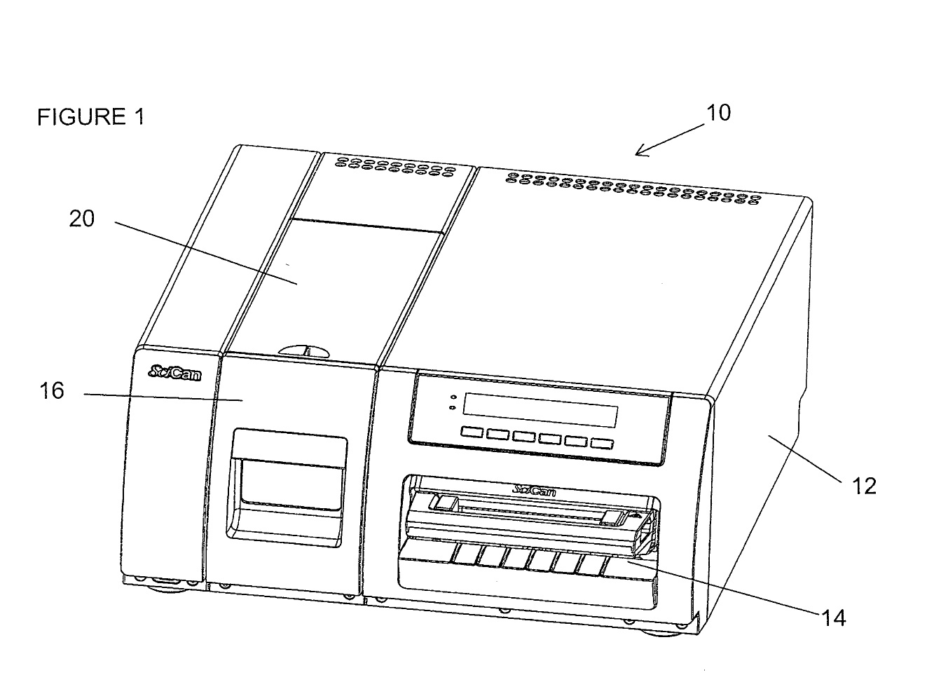

FIG. 1 illustrates an apparatus 10 for sterilizing medical and dental instruments (not shown). The apparatus 10 is shown for illustration purposes as a cassette autoclave, such as that manufactured and sold by the applicant under the trade-mark STATIM, and which is embodied in numerous patents worldwide, including U.S. Pat. No. 5,271,893 of Newman. It is contemplated, however, that the present invention may be suitable for use in other cassette type sterilizers.

As seen in FIG. 1, the apparatus 10 in the illustrated example comprises a cover assembly 12, a cassette assembly 14 and a reservoir 16, with lid 20, in accordance with an embodiment of the present invention. The reservoir 16 is shown inserted into the apparatus 10 in its operable position. The reservoir 16 stores a source of fluid for use by the apparatus 10. Although the reservoir 16 may be used to store and supply any fluid needed for the sterilization process and apparatus 10, the embodiments illustrated below demonstrate a water supply provided in reservoir 16 for the generation of steam by the apparatus 10.

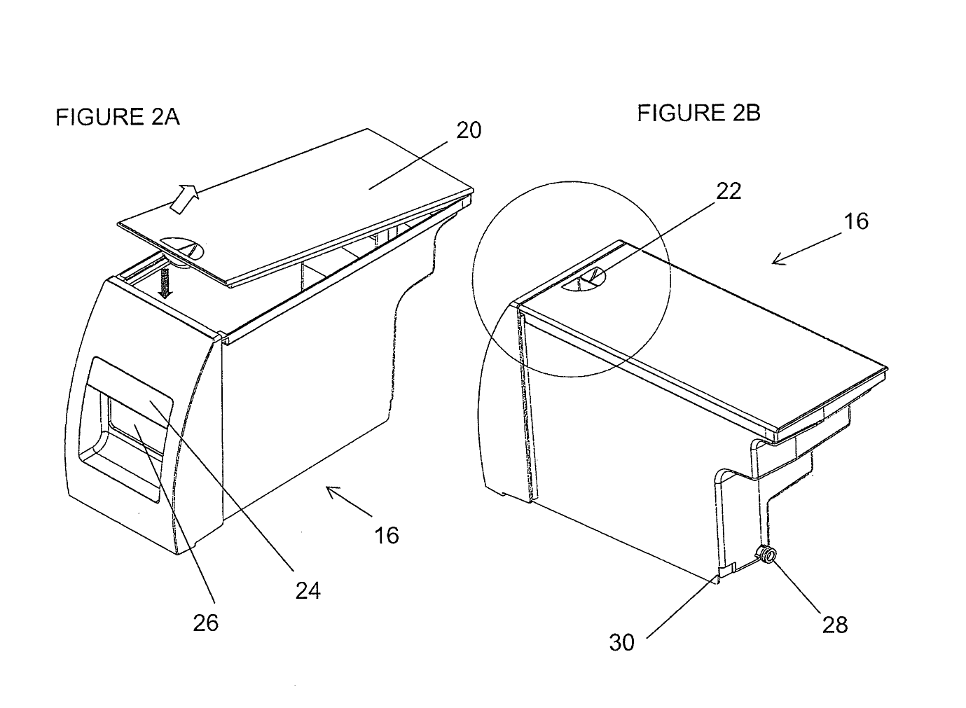

FIGS. 2A and 2B show a reservoir 16 in accordance with an embodiment of the present invention. The reservoir 16 is removable from the apparatus 10 so that it can be carried to a fluid supply, such as a water tap, to be refilled. Reservoir lid 20 is removable to allow the reservoir to be refilled. A recessed grip area 22 in lid 20 enables a user to easily remove the lid 20 when the reservoir 16 is installed in the apparatus 10, or when the reservoir 16 is removed from the apparatus 10. Reservoir 16 also includes a handle 24 for ease of removal of the reservoir 16 from the apparatus 10, as well as a fluid level indicating window 26 to allow a user to see the fluid level within the reservoir 16 when the reservoir 16 is installed in the apparatus 10.

As can be appreciated from FIGS. 1, 2A and 2B reservoir 16 also may be refilled while it is installed in apparatus 10 by removing the lid 20. The reservoir 16 and reservoir lid 20 are preferably manufactured from injection molded plastic such as polycarbonate but any other suitable material and construction for containing fluid in a sterilization apparatus 10 may be used. Furthermore, the injection molded plastic material may be tinted transparent so that the fluid level indicating window 26 can be an integral part of the main body of the reservoir 16. The selection of the appropriate tint minimizes the exposure of the water to certain wavelengths in the direct light spectrum which may otherwise encourage algae growth within the reservoir 16.

As seen in FIG. 2B, a fluid outlet port 28 is provided at the rear of the reservoir 16 for connecting with and providing a supply of fluid to the apparatus 10, via the receiver assembly. In this configuration, the fluid outlet port 28 is shown as a self-closing poppet valve, although other suitable ports or valves may be used. Also located at the rear of the reservoir 16 is a magnet 30 which is used to indicate the presence of the reservoir 16 in the apparatus 10 as described below.

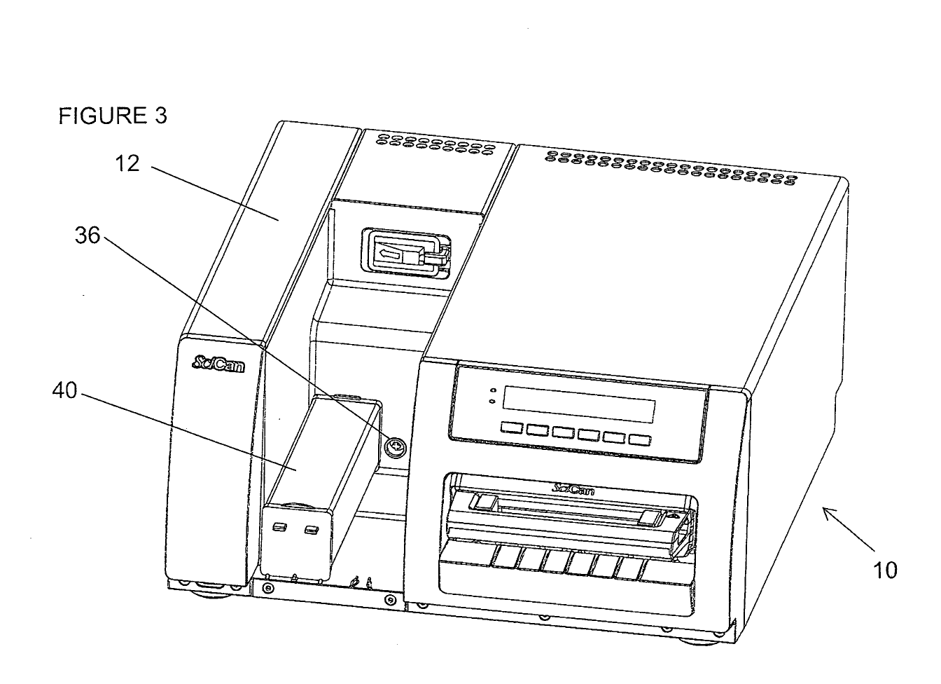

FIG. 3 illustrates the apparatus 10 with the reservoir 16 removed. The receiver assembly, unseen behind the cover assembly 12, provides an inlet port 36 to receive water from the reservoir, via reservoir outlet port 28 as shown in FIG. 2B, for delivery to the apparatus 10. In one embodiment of the present invention, as shown in FIG. 3, a separate cartridge 40, such as a single-use disposable cartridge, may be used and connected to the receiver assembly for further treatment of water from the reservoir 16, if desired.

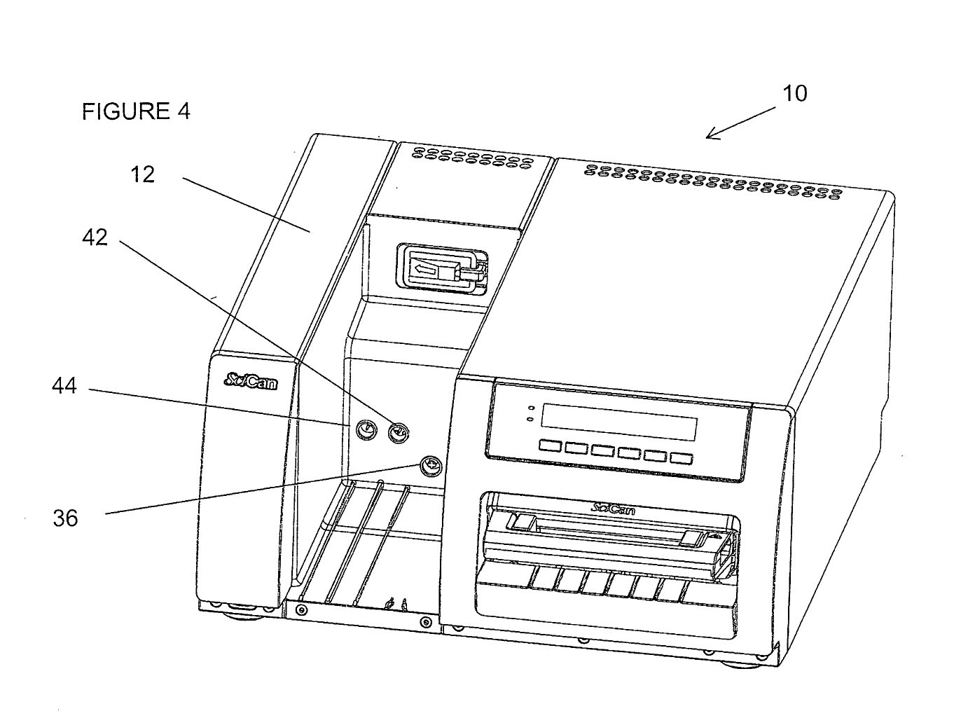

FIG. 4 illustrates the apparatus 10 with the reservoir 16 and cartridge 40 removed. The receiver assembly, unseen behind the cover assembly 12, also provides an outlet port 42 to direct water to the cartridge 40 and a second inlet port 44 to receive water from the cartridge 40 for delivery to the apparatus 10. As described herein in an embodiment of the invention, the cartridge 40 may be a single use disposable cartridge comprising a deionization resin filter for treating water before it is used in the apparatus 10. Other suitable filter means for treating water to be used in a sterilization apparatus 10 may be used in the cartridge 40.

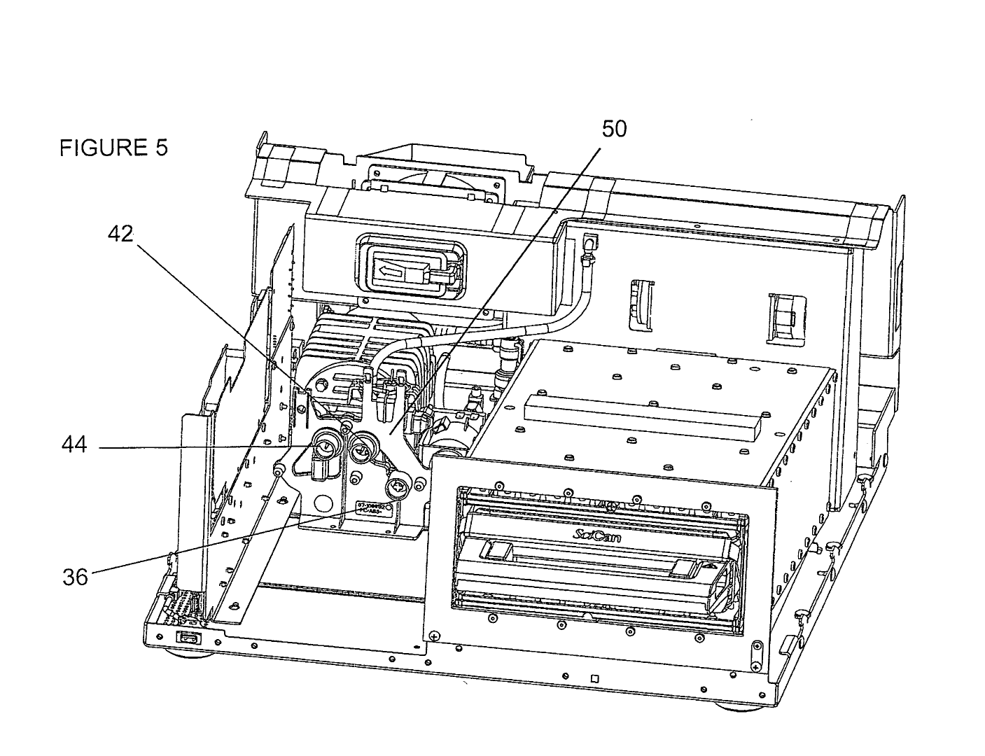

FIG. 5 illustrates the apparatus 10 with the cover assembly 12, reservoir 16 and cartridge 40 removed, showing a receiver assembly 50 in accordance with an embodiment of the present invention. The inlet port 36 for receiving fluid from the reservoir 16, as well as the outlet port 42 and inlet port 44 for connecting to the cartridge 40, are clearly shown on the receiver assembly 50 of FIG. 5.

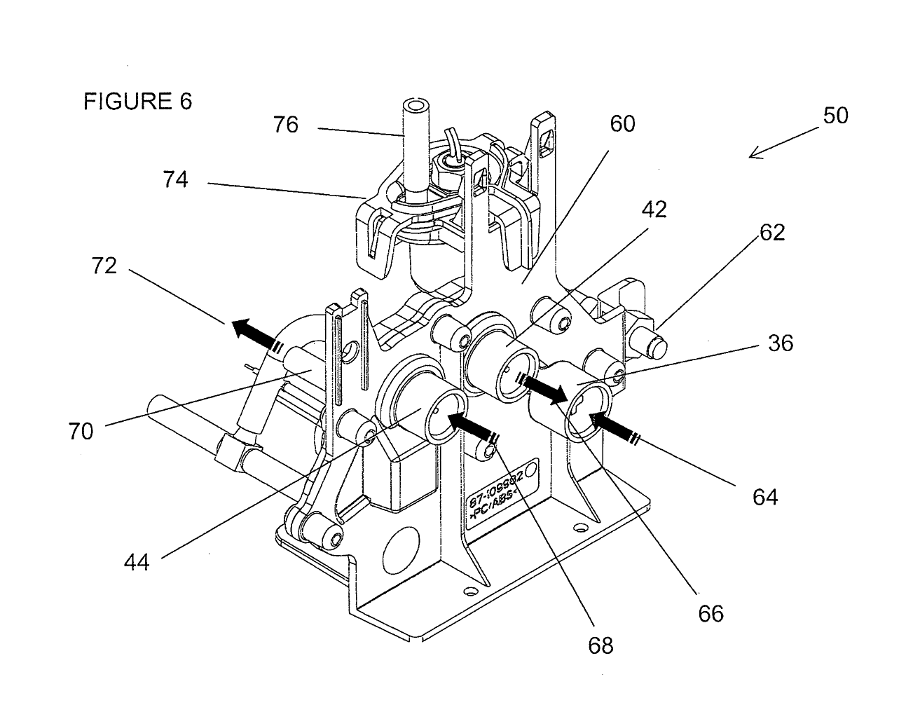

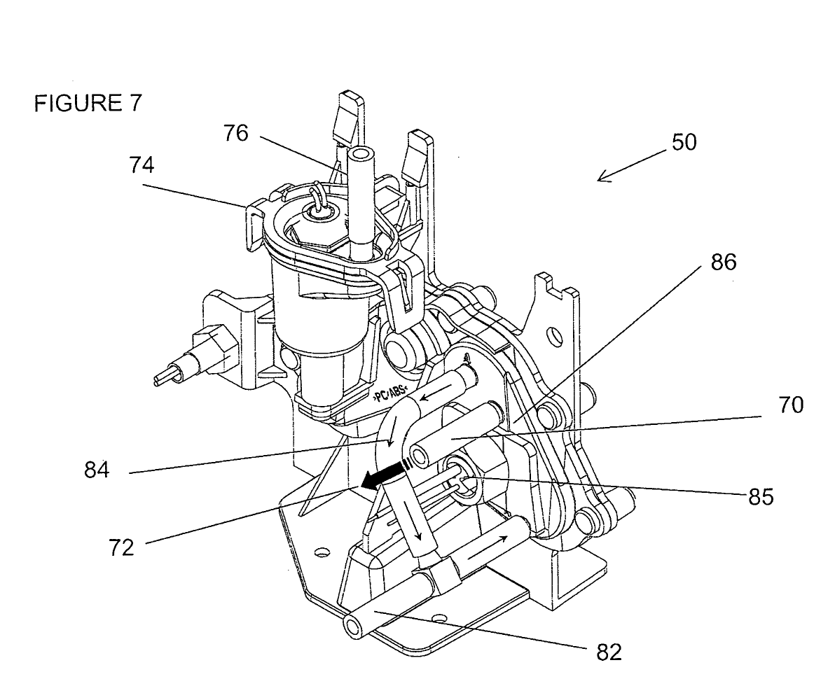

FIGS. 6 and 7 illustrate front and rear views, respectively, of the receiver assembly 50, separated from the apparatus 10. In this configuration, the receiver inlet port 36 and outlet port 42 are shown as self-closing poppet valves although other suitable ports or valves, such as ball and spring valves may be used. The receiver assembly 50 may be constructed from injection molded plastic such as Acrylonitrile butadiene styrene (ABS) or a blend of ABS and polycarbonate (ABS/PC).

As shown in FIG. 6, water from the reservoir 16 enters a first compartment 60 in the receiver assembly 50 through inlet port 36, as shown by arrow 64. In this embodiment, water from the first compartment 60 of receiver assembly 50 is directed to the cartridge 40 via outlet port 42, as shown by arrow 66. Water from the cartridge 40 is received by the receiver assembly 50 through inlet port 44, as shown by arrow 68.

Water is provided from the receiver assembly 50 to the sterilization apparatus 10, via outlet port 70, as shown by arrow 72. In the case of an apparatus such as the STATIM cassette autoclave, water from the receiver assembly 50 is provided to a water pump (not shown). The suction of the water pump and hydrostatic pressure from the water in the reservoir 16 draws the water from the reservoir 16 and through the receiver assembly 50 to the apparatus via outlet port 70.

As seen more clearly in FIG. 7 in the illustrated example, it may be desirable to route the water from the cartridge 40 and inlet port 44 via a tubing 84 to a second compartment 86 in the receiver assembly 50. Tubing 84 may be a silicone tubing or any other suitable material for carrying the sterilization fluid. The outlet port 70 from the second compartment 86 in the receiver assembly 50 then can be located at a level above the inlet port 44 thus ensuring that air, which is lighter than water, will first be flushed out of the second compartment 86 of the receiver assembly 50. As shown in FIG. 7, a normally closed drain tube 82 is provided to allow residual water to be drained from the receiver assembly 50, as required, such as during shipping or maintenance.

FIGS. 6 and 7 also illustrate a fluid level switch assembly 74 and vent tube 76 adjacent to the first compartment 60 and inlet port 36 in the receiver assembly 50. The level of fluid within the switch assembly 74 corresponds to the level of fluid remaining in the reservoir 16, providing an indication to the sterilization apparatus 10 of the status of the fluid supply in reservoir 16.

The receiver assembly 50 may include a conductivity sensor 85 in the second compartment 86 in order to detect the quality of water received at the inlet port 44, such as from a filter housed in cartridge 40.

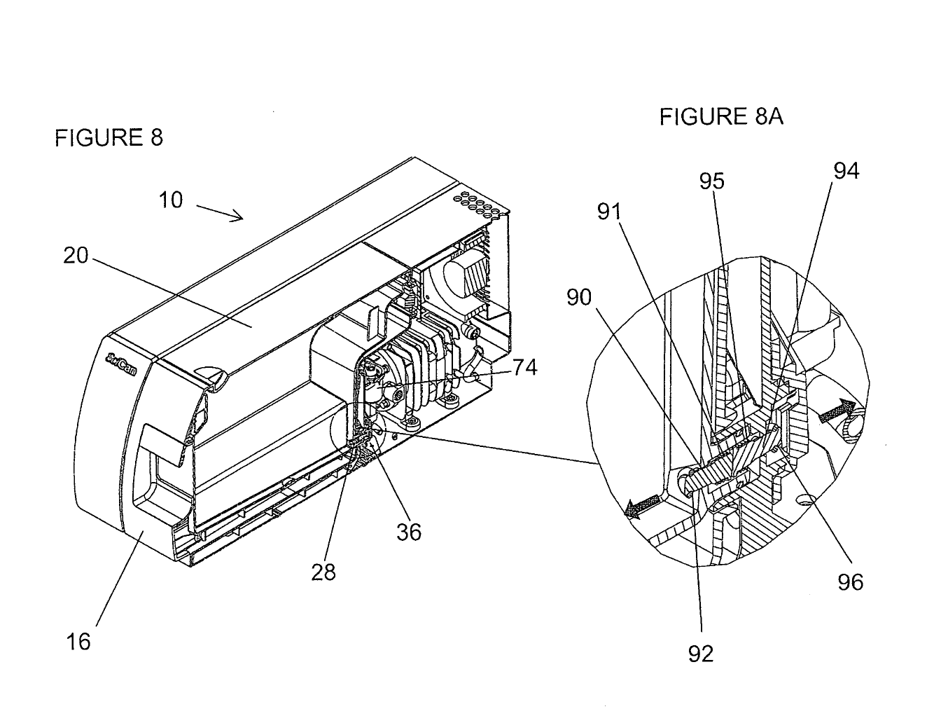

FIGS. 8 and 8A illustrate in further detail the connection of the reservoir 16 to the receiver assembly 50. The reservoir outlet port 28 is aligned to mate with the inlet port 36 of the receiver assembly 50. It is also desirable that the outlet port 28 and inlet port 36 include a means of preventing leakage of any fluid from the reservoir 16 or receiver assembly 50 when the reservoir is removed from the receiver assembly 50 and apparatus 10.

In this illustration, self-closing poppet valves are used to prevent such leakage of fluid. As shown in FIG. 8A a self-closing poppet valve 90, compression spring 91 and o-ring seal 92 are located at the reservoir outlet port 28. A similar self-closing poppet valve 94, compression spring 95 and o-ring seal 96 are located at the inlet port 36 of the receiver assembly 50. As the reservoir 16 is placed in the apparatus 10 and fully engaged with the receiver assembly 50, the poppet valves are pushed open to allow water to flow into the receiver assembly 50. Similarly, the valves 90, 94 close when the reservoir 16 is removed from the apparatus 10 and receiver assembly 50, to prevent leakage of water from the reservoir outlet port 28 and receiver assembly inlet port 36. The leakage prevention means, such as the self-closing poppet valve 94 at the receiver assembly inlet 36, prevents any residual water in the first compartment 60 of the receiver assembly 50 or in the fluid level switch assembly 70 from leaking into the apparatus 10 when the reservoir 16 is removed.

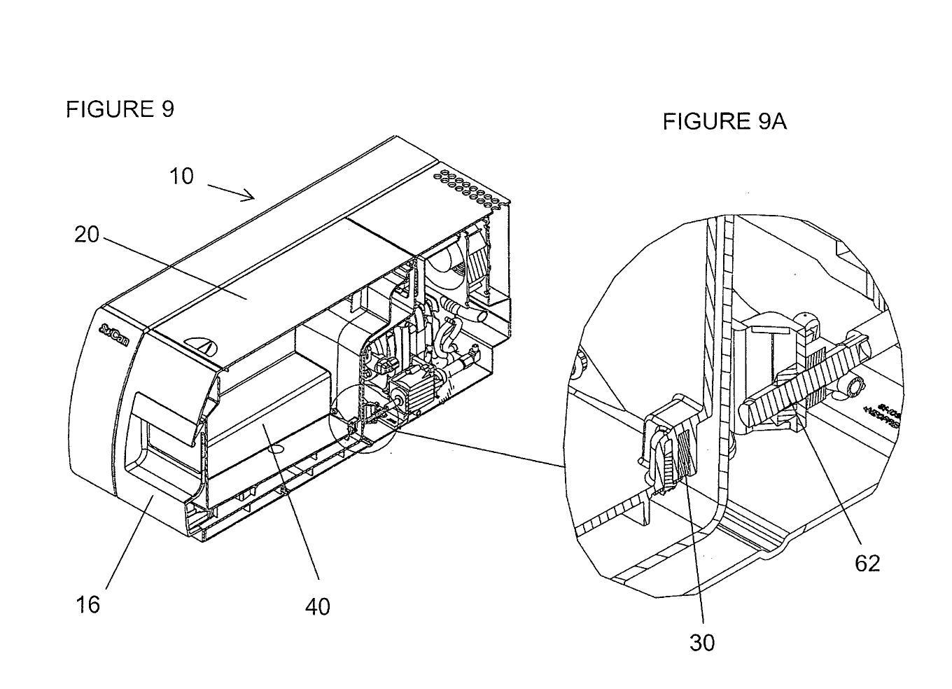

FIGS. 9 and 9A illustrate means for detecting the presence or absence of the reservoir 16 connecting to the receiver assembly 50. This indication may be provided to the sterilization apparatus 10 so that the sterilization apparatus 10 is informed of the presence or absence of the reservoir 16. This can be used by the apparatus 10 to, for example, prevent the start of or halt the sterilization process if the fluid supply is absent.

As illustrated in FIG. 9A, and as also shown in FIG. 2B, an indicator such as a magnet 30 is located at the rear of the reservoir 16. A corresponding means for detecting the reservoir 16 is located on the receiver assembly 50. As shown in FIG. 9A and FIG. 6, a proximity switch 62, such as a reed switch, is located on the receiver assembly 50 so as to be aligned with the magnet 30 when the reservoir 16 is fully engaged with the receiver assembly 50 and apparatus 10. When the reservoir 16 is properly inserted in the apparatus 10, the proximity switch is activated to indicate the presence of the reservoir 16. Such indication means alerts the apparatus 10 to the presence of the reservoir 16 without requiring additional sensors or openings within the reservoir 16. The indication and detection means have been illustrated as a magnet and reed switch, but other suitable indication and detection means may be used.

In order to prevent any leakage of fluid within the apparatus 10 and to ensure a proper supply of fluid or water from reservoir 16 to the apparatus 10 via the receiver assembly 50, it is desirable to ensure the reservoir 16 and cartridge 40 are both present and properly inserted into apparatus 10.

As discussed above, in one embodiment of the present invention, the receiver assembly 50 is configured with inlet and outlet ports 42, 44 to provide and receive fluid from a cartridge 40. The treated fluid, such as filtered water, is then provided to the apparatus 10. As shown in FIG. 3, the cartridge 40 may be located in the apparatus 10 adjacent to and beneath the reservoir 16. The presence or absence of the cartridge 40 in the apparatus 10 is indicated by the ability to properly insert the reservoir 16 in receiver assembly 50.

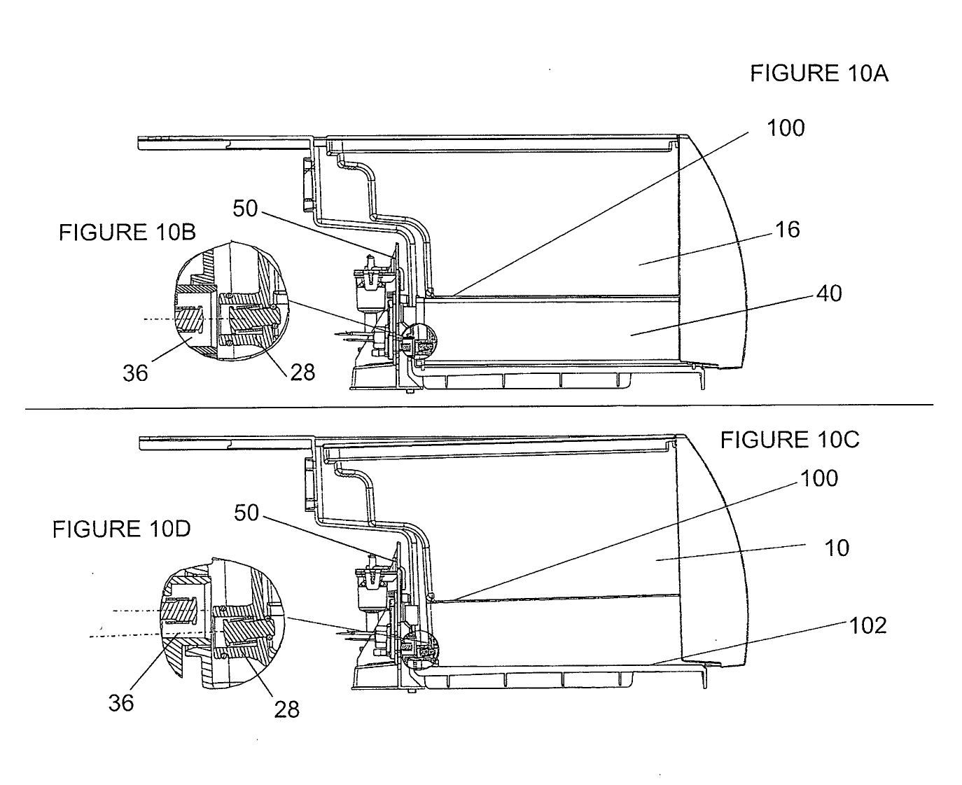

FIGS. 10A and 10B illustrate means for detecting the presence or absence of the cartridge 40 connecting to the receiver assembly 50. This indication may be provided to the end user so that the end user is informed of the presence or absence of the cartridge 40. FIG. 10A illustrates the presence of a cartridge 40 and the reservoir 16 in the apparatus 10. Ribs 100 along the bottom of the reservoir 16 slide along the surface of the cartridge 40 as the reservoir 16 is inserted in the apparatus 10. As seen in further detail in FIG. 10B, the presence of cartridge 40 ensures the proper alignment of the outlet port 28 of the reservoir 16 and the inlet port 36 of the receiver assembly 50.

As shown in FIGS. 10C and 10D, in the absence of the cartridge 40, the water reservoir 16 would rest at an angle along the compartment floor 102 of the apparatus 10. As seen in further detail in FIG. 10D, misalignment of the outlet port 28 of the reservoir 16 and the inlet port 36 of the receiver assembly 50 would result and the apparatus 10 would detect and indicate the absence of reservoir 16, alerting the user to problems with the cartridge 40.

In one embodiment of the present invention, reservoir 16 and receiver assembly 50 may be configured to accept a cartridge 40 in order to provide a treated supply of water to the sterilization apparatus 10. However, it can be appreciated that where treated water, such as distilled or deionized water, is used in the reservoir 16, a cartridge 40 is not necessary to further treat the water supplied to the apparatus 10.

In this case, a cartridge 40 containing a simple bypass routing water or fluid received from the receiver outlet port 42 to the inlet port 44 may be used with the receiver assembly 50 according to one embodiment of the present invention. The cartridge 40 containing a bypass also would provide the alignment means to ensure the reservoir 16 is properly aligned with the receiver assembly 50 as illustrated in FIGS. 10A and 10B.

Alternatively, the receiver assembly 50 may consist of a single inlet port for receiving treated water from the reservoir 16 and a single outlet port for directed treated water to the apparatus 10. In this case, reservoir 16 may be configured to align with the receiver assembly 50 and apparatus 10 in the absence of a cartridge. In a further embodiment, the receiver assembly may comprise a single inlet port and a single outlet port and include a filter to treat the fluid from the reservoir 16, such as tap water, before supplying the treated fluid to the sterilization apparatus 10.

It will be appreciated by those skilled in the art that a filter or similar water treatment means also may be housed within the reservoir 16 itself or within the first and/or second compartments 60, 86 of the receiver assembly 50 in order to filter or treat the water before it is used in the sterilization steps.

Thus, it is apparent that there has been provided in accordance with the invention a receiver assembly and reservoir for use in a sterilization apparatus that fully satisfy the objects, aims and advantages set forth above. While the invention has been described in conjunction with illustrated embodiments thereof, it is evident that many alternatives, modifications and variations will be apparent to those skilled in the art in light of the foregoing description. Accordingly, it is intended to embrace all such alternatives, modifications and variations as fall within the spirit and broad scope of the invention.

1. A fluid supply assembly for use in a sterilization apparatus, said assembly comprising:
a removable fluid reservoir having a fill means and an outlet port; and
an interface assembly having at least one inlet for receiving fluid from said reservoir and at least one outlet for directing fluid to the sterilization apparatus.

2. The fluid supply assembly of claim 1 wherein said removable fluid reservoir further includes filter means.

3. The fluid supply assembly of claim 1 wherein said interface assembly further includes filter means.

4. The fluid supply assembly of claim 1 further comprising a fluid cartridge housing filter means.

5. The fluid supply assembly of claim 4 wherein the interface assembly further comprises a filter outlet for directing fluid to said filter means and a filter inlet for receiving fluid from said filter means.

6. The fluid supply assembly of claim 4 further comprising fluid cartridge detection means.

7. The fluid supply assembly of claim 4 wherein said removable fluid reservoir outlet port and said interface assembly inlet further include means to prevent leakage of fluid when said removable fluid reservoir is removed.

8. The fluid supply assembly of claim 7 wherein the means to prevent leakage of fluid includes self-closing valve means at said removable fluid reservoir outlet port and at said interface assembly inlet.

9. The fluid supply assembly of claim 4 further comprising fluid reservoir detection means.

10. The fluid supply assembly of claim 9 wherein said removable fluid reservoir detection means comprises a reed switch located on said interface assembly and a magnet located on said removable fluid reservoir.

11. The fluid supply assembly of claim 1 further comprising fluid level detection means.

12. The fluid supply assembly of claim 1 wherein said interface assembly further includes a conductivity sensor.

13. A fluid supply assembly for use in a sterilization apparatus, said assembly comprising:
a removable fluid reservoir having a fill means and an outlet port;
a fluid cartridge having an inlet and an outlet; and
an interface assembly having a first inlet for receiving fluid from said removable fluid reservoir, a first outlet for directing fluid to said fluid cartridge, a second inlet for receiving fluid from said fluid cartridge and a second outlet for directing fluid to the sterilization apparatus.

14. The fluid supply assembly of claim 13 wherein said fluid cartridge comprises a filter.

15. The fluid supply assembly of claim 13 wherein said fluid cartridge comprises a fluid bypass.

16. The fluid supply assembly of claim 13 wherein said removable fluid reservoir further includes filter means for treating water.

17. The fluid supply assembly of claim 13 further comprising fluid cartridge detection means.

18. The fluid supply assembly of claim 13 wherein said removable fluid reservoir outlet port and said interface assembly inlet further include means to prevent leakage of fluid when the removable fluid reservoir is removed.

19. The fluid supply assembly of claim 18 wherein said means to prevent leakage of fluid includes self-closing valve means at said removable fluid reservoir outlet port and at said interface assembly inlet.

20. The fluid supply assembly of claim 13 further comprising removable fluid reservoir detection means.

21. The fluid supply assembly of claim 20 wherein said fluid reservoir detection means comprises a reed switch located on the interface assembly and a magnet located on the removable fluid reservoir.

22. The fluid supply assembly of claim 13 further comprising fluid level detection means.

23. The fluid supply assembly of claim 13 wherein said interface assembly further includes a conductivity sensor.
